(19) United States
(12) Patent Application Publication
(10) Pub. No.: US 2020/0041395 A1
(43) Pub. Date: Feb. 6, 2020

(54) IDENTIFICATION OF FLUID PROPERTIES USING A PIEZO HELM RESONATOR

(71) Applicant: Baker Hughes Oilfield Operations LLC, Houston, TX (US)

(72) Inventors: Dwight Swett, Houston, TX (US); Otto Fanini, Houston, TX (US)

(73) Assignee: Baker Hughes Oilfield Operations LLC, Houston, TX (US)

(21) Appl. No.: 16/051,022

(22) Filed: Jul. 31, 2018

(51) Int. Cl. G01N 9/00; E21B 49/08; G01N 9/36; G01N 29/036; G01N 29/24; G01N 29/34; G01N 33/28

(52) U.S. Cl. CPC G01N 9/002; E21B 49/08; G01N 9/36; G01N 33/2823; G01N 29/2437; G01N 29/34; G01N 29/036

(57) ABSTRACT

Embodiments of the present disclosure include a method for determining at least one fluid property that includes obtaining electrical admittance data from a downhole tool, the admittance data being associated with a fluid in a wellbore. The method also includes obtaining reference electrical admittance data for air. The method further includes comparing the admittance data to the reference admittance data. The method also includes determining an offset between a first peak of the admittance data and a second peak of the reference admittance data. The method includes determining the at least one fluid property based at least in part on the determined resonance frequency offset.

### BACKGROUND

### 1. Field of the Invention

The present disclosure relates to downhole measurement devices. More particularly, the present disclosure relates to identification of fluid properties using downhole measurement devices.

### 2. Description of Related Art

During oil and gas operations, it is often difficult to determine fluid properties in a downhole well due to inaccessibility, contamination of fluids, mixing of fluids, and the like. As a result, typical operations deploy multiple tools that may be specialized to determine a single fluid property, such as density. These tools are often fragile, and as a result, may not be utilized in multiple operations. Furthermore, installing multiple tools along a drill or wireline string increases costs of the operation and also may lead to slower drilling and or wireline logging operations because some tools are individually tripped into and out of the well.

### SUMMARY

Applicants recognized the problems noted above herein and conceived and developed embodiments of systems and methods, according to the present disclosure, for a piezo helm resonator for identification of fluid properties.

In an embodiment a system for measuring a fluidic property of a fluid includes a housing including an opening, the opening extending longitudinally along an axis of the housing. The system also includes a flow passage extending through the passage, the flow passage intersecting the opening. The system further includes a flow diverter arranged at an intersection between the opening and the flow passage, the flow diverter directing a fluid flowing through the flow passage into a fluid cavity formed at least partially in the opening. The system also includes a piezo helm resonator arranged within the fluid cavity, the piezo helm resonator electrically coupled to a power supply that transmits electrical energy to at least one resonator electrode arranged on the piezo helm resonator, wherein the piezo helm resonator resonates within the fluid cavity when electrically energized by the power supply.

In another embodiment a method for determining a fluid property includes positioning a helm resonator sensor within a wellbore. The method also includes directing a flow of fluid into a fluid cavity of the helm resonator sensor. The method further includes transmitting electrical energy to a piezo helm resonator within the fluid cavity. The method also includes collecting data associated with at least one fluid property via the piezo helm resonator. The method includes determining the at least one fluid property based at least in part on the collected data.

In an embodiment a method for determining at least one fluid property includes obtaining electrical admittance data from a downhole tool, the electrical admittance data being associated with a fluid in a wellbore. The method also includes obtaining reference electrical admittance data for a reference fluid. The method further includes comparing the admittance data to the reference admittance data. The method also includes determining a set of admittance resonance frequency offsets from the reference admittance data. The method includes determining the at least one fluid property based at least in part on the determined set of offsets.

### BRIEF DESCRIPTION OF DRAWINGS

The foregoing aspects, features, and advantages of the present disclosure will be further appreciated when considered with reference to the following description of embodiments and accompanying drawings. In describing the embodiments of the disclosure illustrated in the appended drawings, specific terminology will be used for the sake of clarity. However, the disclosure is not intended to be limited to the specific terms used, and it is to be understood that each specific term includes equivalents that operate in a similar manner to accomplish a similar purpose.

FIG. 1 is a schematic side view of an embodiment of a wireline system, in accordance with embodiments of the present disclosure;

FIG. 2 is an isometric view of an embodiment of a helm resonator sensor, in accordance with embodiments of the present disclosure;

FIG. 3 is an isometric view of an embodiment of a helm resonator sensor, in accordance with embodiments of the present disclosure;

FIG. 4 is a cross-sectional exploded view of an embodiment of a helm resonator sensor, in accordance with embodiments of the present disclosure;

FIG. 5 is a cross-sectional isometric view of an embodiment of a helm resonator sensor, in accordance with embodiments of the present disclosure;

FIG. 6 is a cross-sectional side view of an embodiment of a helm resonator sensor, in accordance with embodiments of the present disclosure;

FIG. 7 is a cross-sectional side view of an embodiment of a helm resonator sensor, in accordance with embodiments of the present disclosure;

FIG. 8 is a top plan view of an embodiment of a piezo helm resonator within a fluid cavity, in accordance with embodiments of the present disclosure;

FIG. 9 is an isometric view of an embodiment of a piezo helm resonator, in accordance with embodiments of the present disclosure;

FIG. 10 is a graphical representation of an embodiment of an electrical admittance magnitude spectrum for determining density, in accordance with embodiments of the present disclosure;

FIG. 11 is a graphical representation of an embodiment of a set of electrical admittance real and imaginary components spectra for determining viscosity, in accordance with embodiments of the present disclosure;

FIG. 12 is a graphical representation of an embodiment of an electrical admittance phase spectrum for determining viscosity, in accordance with embodiments of the present disclosure;

FIG. 13 is a schematic diagram of an embodiment of a machine learning system, in accordance with embodiments of the present disclosure;

FIG. 14 is a flow chart of an embodiment of a method for determining fluid properties, in accordance with embodiments of the present disclosure; and

FIG. 15 is a flow chart of an embodiment of a method for determining a fluid characterization using a machine learning system, in accordance with embodiments of the present disclosure.

### DETAILED DESCRIPTION

The foregoing aspects, features, and advantages of the present disclosure will be further appreciated when considered with reference to the following description of embodiments and accompanying drawings. In describing the embodiments of the disclosure illustrated in the appended drawings, specific terminology will be used for the sake of clarity. However, the disclosure is not intended to be limited to the specific terms used, and it is to be understood that each specific term includes equivalents that operate in a similar manner to accomplish a similar purpose.

When introducing elements of various embodiments of the present disclosure, the articles “a”, “an”, “the”, and “said” are intended to mean that there are one or more of the elements. The terms “comprising”, “including”, and “having” are intended to be inclusive and mean that there may be additional elements other than the listed elements. Any examples of operating parameters and/or environmental conditions are not exclusive of other parameters/conditions of the disclosed embodiments. Additionally, it should be understood that references to “one embodiment”, “an embodiment”, “certain embodiments”, or “other embodiments” of the present disclosure are not intended to be interpreted as excluding the existence of additional embodiments that also incorporate the recited features. Furthermore, reference to terms such as “above”, “below”, “upper”, “lower”, “side”, “front”, “back”, or other terms regarding orientation or direction are made with reference to the illustrated embodiments and are not intended to be limiting or exclude other orientations or directions.

Embodiments of the present disclosure include a helm resonator sensor that receives a flow of fluid for interrogation within a fluid cavity using a piezo helm resonator. In various embodiments, the piezo helm resonator receives electrical energy to resonator electrodes arranged on opposing faces of the piezo helm resonator. The electrical energy induces a strain in the direction of opposing electrode faces across the thickness of a strain bar of the piezo helm resonator, which drives longitudinal displacement along a transverse (e.g., cross) axis relative to the opposing electrodes direction. This longitudinal displacement may lead to a resonance response that, when the piezo helm resonator is surrounded by fluid within the fluid cavity, enables an electrical admittance spectrum to be measured. In various embodiments, the electrical admittance may be compared against a reference electrical admittance, for example an air fluid electrical admittance, and a set of offsets may be determined to calculated one or more fluid properties, such as density, viscosity, or the like. Furthermore, in various embodiments, the piezo helm resonator may also include electromagnetic spectroscopy coils to enable measurements of electrical conductivity within the fluid cavity. In certain embodiments, one or more machine learning systems may be utilized in order to classify fluid compositions, for example based on a contamination. For example, contamination may refer to a percentage of fluid composition that is not hydrocarbons. The machine learning system may receive input data corresponding to properties such as density, viscosity, conductivity, and the like for a variety of different fluid classifications. The machine learning system may then be used to correlate the data to data obtained from the piezo helm resonator. In this manner, a variety of different fluid properties or fluid classifications may be determined using a single downhole sensor.

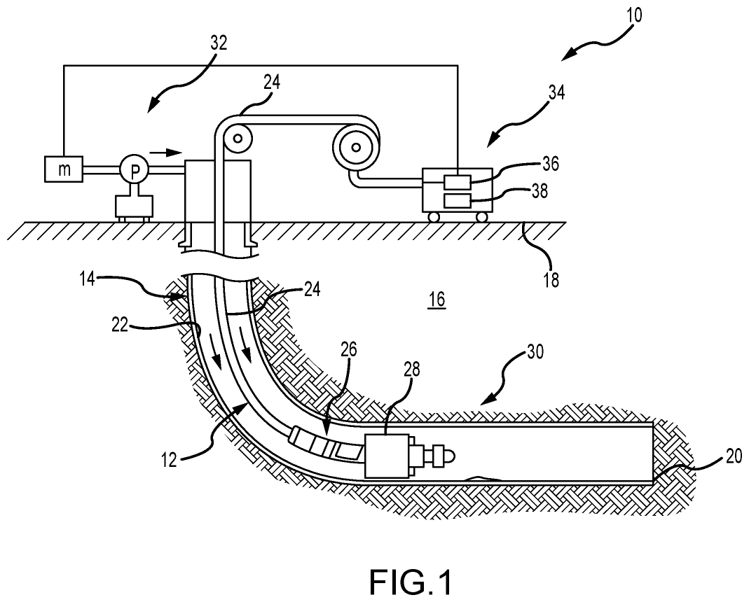

FIG. 1 is a schematic elevation view of an embodiment of a wellbore system 10 that includes a work string 12 shown conveyed in a wellbore 14 formed in a formation 16 from a surface location 18 to a depth 20. The wellbore 14 is shown lined with a casing 22, however it should be appreciated that in other embodiments the wellbore 14 may not be cased. In various embodiments, the work string 12 includes a conveying member 24, such as an electric wireline, and a downhole tool or assembly 26 (also referred to as the bottomhole assembly or “BHA”) attached to the bottom end of the wireline. The illustrated downhole assembly 26 includes various tools, sensors, measurement devices, communication devices, and the like, which will not all be described for clarity. In various embodiments, the downhole assembly 26 includes a measurement module 28, which will be described below, determining one or more properties of the formation 16. In the illustrated embodiment, the downhole tool 28 is arranged in a horizontal or deviated portion 30 of the wellbore 14, however it should be appreciated that the downhole tool 28 may also be deployed in substantially vertical segments of the wellbore 14.

The illustrated embodiment further includes a fluid pumping system 32 at the surface 18 that includes a motor that drives a pump to pump a fluid from a source into the wellbore 14 via a supply line or conduit. To control the rate of travel of the downhole assembly, tension on the wireline 14 is controlled at a winch on the surface. Thus, the combination of the fluid flow rate and the tension on the wireline may contribute to the travel rate or rate of penetration of the downhole assembly 16 into the wellbore 14. The wireline 14 may be an armored cable that includes conductors for supplying electrical energy (power) to downhole devices and communication links for providing two-way communication between the downhole tool and surface devices. In aspects, a controller 34 at the surface is provided to control the operation of the pump and the winch to control the fluid flow rate into the wellbore and the tension on the wireline 12. In aspects, the controller 34 may be a computer-based system that may include a processor 36, such as a microprocessor, a storage device 38, such as a memory device, and programs and instructions, accessible to the processor for executing the instructions utilizing the data stored in the memory 38.

As described above, the illustrated embodiment includes the measurement module 28. As will be described below, in various embodiments, the measurement module 28 may include one or more piezo helm resonators for determination of various fluid properties within the wellbore 14. For example, oil and gas products may enter an annulus and flow along the BHA 26. At least a portion of that flow may be redirected into the measurement module 28. Within the measurement module 28, or proximate the measurement module 28 in certain embodiments, one or more fluid properties may be measured to facilitate wellbore operations. Furthermore, it should be appreciated that while various embodiments include the measurement module 28 incorporated into a wireline system, in other embodiments the measurement module 28 may be associated with rigid drill pipe, coiled tubing, or any other downhole exploration and production method.

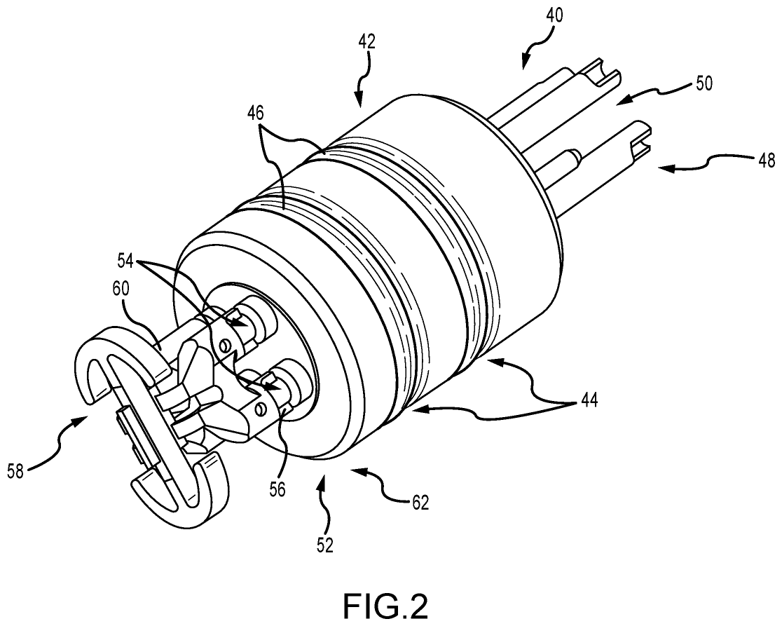

FIG. 2 is a front perspective view of an embodiment of a helm resonator sensor 40. The helm resonator sensor 40 may be deployed with the drill string 14, for example via the BHA 24 and/or the measurement module 32, to determine one or more fluid properties within the wellbore 18. The illustrated helm resonator sensor 40 includes a housing 42 having grooves 44 that receive seals 46. In various embodiments, the seals 46 are annular and fit within the annular grooves 44 for forming a substantially liquid-tight seal between the helm housing 42 and a surrounding tubular, such as a tubular within the measurement module 32. As will be described below, the helm resonator sensor 40 may be arranged proximate a fluid cavity, and the seals 46 may be used to form at least a portion of the cavity. The housing includes a first end 48 having openings (not shown) for receiving one or more cables 50, which may provide electrical power to one or more components associated with the helm resonator sensor 40. It should be appreciated that the cables 50, electrodes 56, and electrode lugs 60 may be coated (as illustrated) for protection and insulation. A second end 52 includes openings 54 though which one or more conductors or electrodes 56 extend. The electrodes 56 transmit electrical energy from the cables 50 to a piezo helm resonator 58, which may be supported by the electrode lug structure 60.

In the illustrated embodiment, the electrode lug structure 60 extends axially away from the housing 42, thereby forming a gap 62 between the housing and the piezo helm resonator 58. As will be described below, the gap 62 may be utilized to enable a fluid (e.g., gas, liquid, solid particles, or combination thereof) to flow over and around the piezo helm resonator 58. The piezo helm resonator 58 is secured to the electrode lug structure 60 along a central portion, which will be described in more detail below, and receives electrical energy from the electrodes 56. In various embodiments, the electrical energy transmitted from the electrodes 56 induces a vibration within the piezo helm resonator 58, for example due to resonant displacement as a result of electrodes arranged on the piezo helm resonator. This vibration may be utilized to measure one or more properties of fluid surrounding and/or flowing along the piezo helm resonator 58.

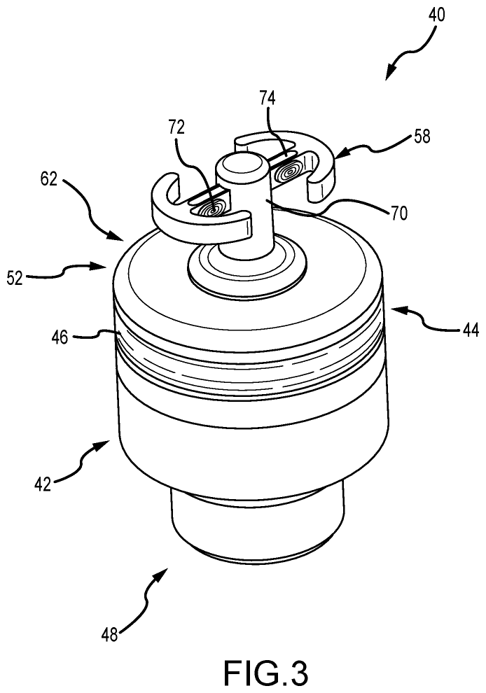

FIG. 3 is a perspective view of an embodiment of the helm resonator sensor 40 in which the electrode lug structure 60 and electrodes 56 are encapsulated with an additional electrically insulating elastomer boot 70 acting to augment the electrical insulation coating. It should be appreciated that the boot 70 may also be utilized with the embodiment shown in FIG. 2. In various embodiments, as will be described below, the piezo helm resonator 58 includes various structures for performing measurements, such as an electromagnetic spectroscopy coil 72, a resonator electrode 74, and the like. As will be described herein, the electromagnetic spectroscopy coil 72 may enable measurement of fluid electrical conductivity using the helm resonator sensor 40. Furthermore, the resonator electrode 74 may be used to determine fluid density and viscosity. Additionally, in various embodiments, a contamination may be determined using a combination of measurements to thereby determine a quality of the fluid (e.g., proportion of fluid that is hydrocarbon as compared to other fluids such as drilling mud or fracturing fluid) in order to assess production levels of the well.

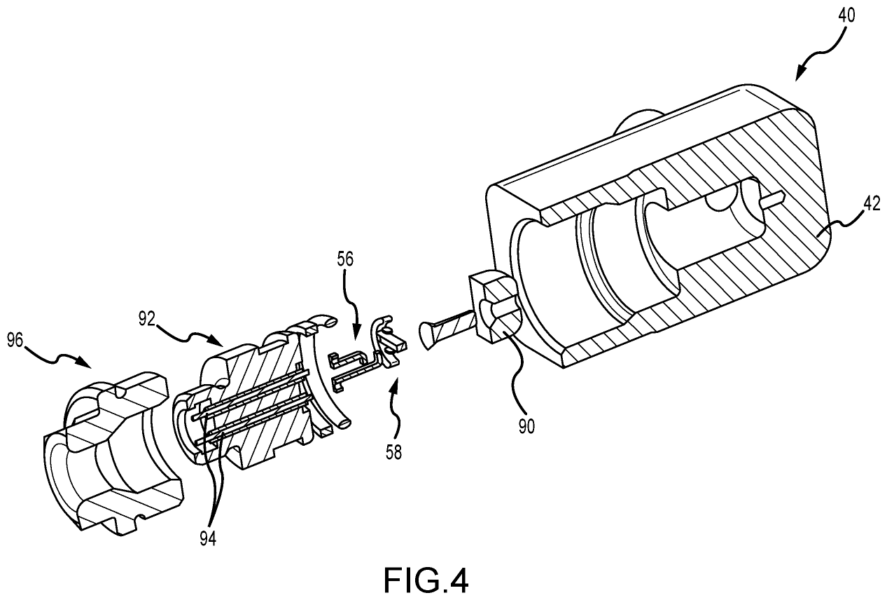

FIG. 4 is a cross-sectional isometric exploded view of an embodiment of the helm resonator sensor 40. The embodiment illustrated in FIG. 4 shows the housing 42, which receives a flow diverter 90 for circulating fluid around the piezo helm resonator 58, which will be described in more detail below. The illustrated piezo helm resonator 58 is coupled to the electrode 56 in the illustrated embodiment, however, as discussed above, the support structure 60 may also be incorporated to maintain the gap 62 between the piezo helm resonator 58 and the housing 42. The housing 42 further receives a feedthrough 92, which includes channels 94 for the cables 50. The cables 50 extend through the channels 94 and couple to the electrode 56, thereby transmitting electrical energy to the piezo helm resonator 58. Also illustrated is a retainer 96, which secures the components within the housing 42. In various embodiments, the retainer 96 includes coupling members, such as threads or the like, which may mate with matching coupling members of the housing 42 to secure the retainer 96 to the housing 42.

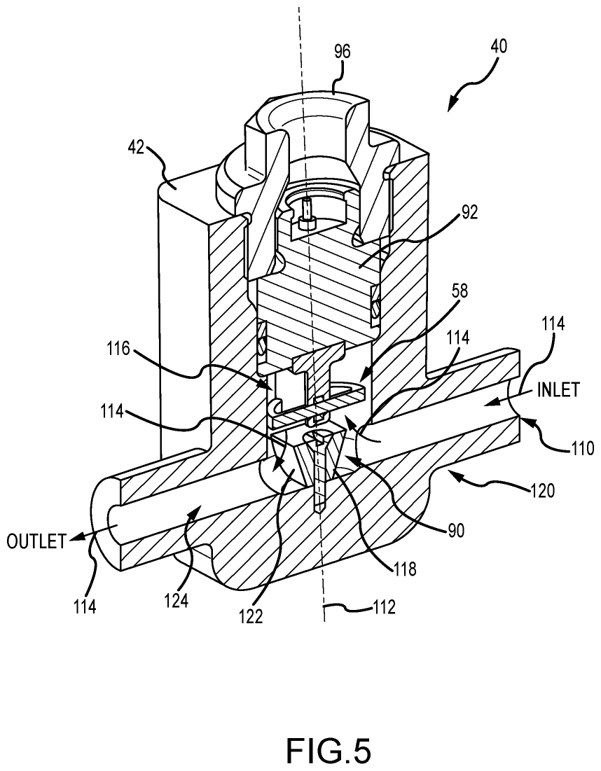

FIG. 5 is an isometric cross-sectional view of an embodiment of the helm resonator sensor 40 in fluid communication with a flow line 110. In various embodiments, the flow line 110 may receive fluid from the annulus 22 for directing the fluid into the measurement module 32 and/or the BHA 24. However, it should be appreciated that the flow line 110 may not necessarily be arranged substantially perpendicular to an axis 112 of the helm resonator sensor 40, and may, in various embodiments, be arranged in different configurations that enable the fluid to substantially surround the piezo helm resonator 58. In the illustrated embodiment, the flow line 110 couples to the housing 42, for example to one or more inlet or outlet ports formed in the housing 42 for receiving the flow line 110. Furthermore, as will be described, the flow line 110 enables the fluid surrounding the piezo helm resonator 58 to be renewed or circulated via the flow diverter 90. In the illustrated embodiment, the flow diverter 90 extends at least partially into the flow line 110 and directs a flow of fluid, represented by arrow 114, into a fluid cavity 116. The piezo helm resonator 58 is arranged within the fluid the cavity 116, and as a result, is exposed to the fluid within the fluid cavity 116.

In the illustrated embodiment, the fluid diverter 90 has a leading edge 118 arranged to face an upstream portion 120 of the flow line 110, and a trailing edge 122 arranged to face a downstream portion 124 of the flow line 110. In operation, as the flow of fluid 114 moves in a downstream direction through the flow line 110, the fluid will contact the leading edge 118 and be driven into the fluid cavity 116. The continued flow of fluid through the flow line 110 will drive the fluid in the fluid cavity 116 out of the fluid cavity 116 along the trailing edge 122 and through the downstream portion 124 of the flow line 110. Advantageously, this will enable different segments of flow to be evaluated by the helm resonator sensor 40. For example, rather than having fluid pool within the fluid cavity 116, the flow diverter 90 will continuously enable the fluid cavity 116 to be flushed, thereby providing measurements for fluid at different stages of flow through the well. As will be described below, in various embodiments a gap thickness is arranged between walls of the fluid cavity 116 and the piezo helm resonator 58, thereby enabling the circulating fluid flow.

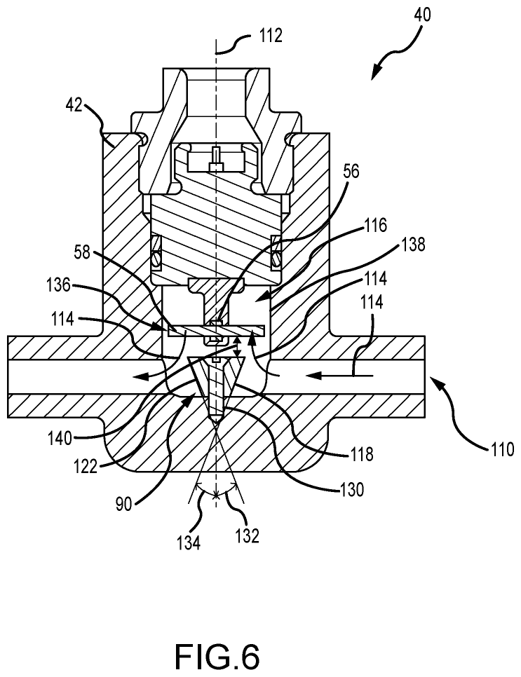

FIG. 6 is a schematic cross-sectional view of an embodiment of the helm resonator sensor 40 installed such that the flow diverter 90 is arranged within the flow line 110. In the illustrated embodiment, the fluid diverter 90 extends into a groove 130 formed in the housing 42. In various embodiments, the flow diverter 90 includes seals or the like to create a fluid tight seal at the groove 130, thereby blocking fluid from bypassing flow into the fluid cavity 116.

In various embodiments, the leading edge 118 and the training edge 122 are arranged at angles 132, 134 with respect to the axis 112. It should be appreciated that the angles 132, 134 may be equal or different and further may be particularly selected based on a variety of conditions, such as flow line size, fluid cavity size, expected flow rate, and the like. In the illustrated embodiment, the angles 132, 134 are approximately 20 degrees. However, in other embodiments, the angles 132, 134 may be approximately 15 degrees, approximately 25 degrees, approximately 30 degrees, approximately 35 degrees, approximately 40 degrees, or any other reasonable angle. Further, the angles 132, 134 may be within particularly selected ranges, such as between 10 degrees and 30 degrees, between 20 degrees and 40 degrees, between 30 degrees, and 50 degrees, and any other reasonable range. In this manner, flow into the fluid cavity 116 may be controlled.

In various embodiments, the flow diverter 90 is formed from a low acoustic impedance material, such as PEEK. By forming the flow diverter 90 from a low acoustic impedance material, anomalous admittance distortion due to acoustic reflections from the flow diverter may be reduced or eliminated. However, forming the flow diverter 90 from low acoustic impedance material reduces the likelihood of admittance distortion and reduces resonance frequency spectra contamination so that improved measurements are made by the helm resonator sensor 40.

As illustrated in FIG. 6, the piezo helm resonator 58 is supported within the fluid cavity 116 by the electrode 56. A gap thickness 136 is formed between an annular wall 138 of the fluid cavity 116 and the piezo helm resonator 58. In various embodiments, the gap thickness 136 is substantially uniform around the piezo helm resonator 58. In other words, the piezo helm resonator 58 may be substantially centered within the fluid cavity 116. Furthermore, in the illustrated embodiment, the piezo helm resonator 58 is arranged a distance 140 from the flow diverter 90. It should be appreciated that the distance 140 may be particularly selected based on operating conditions. In various embodiments, the fluid cavity 116 may have an active volume that is approximately twice a thickness of the piezo helm resonator 58. The active volume may refer to the quantity of fluid that substantially surrounds the piezo helm resonator 58 and which is interrogated to obtain information such as density, viscosity, conductivity, and the like. The active volume may be at least partially considered when determining the distance 140, among other measurements.

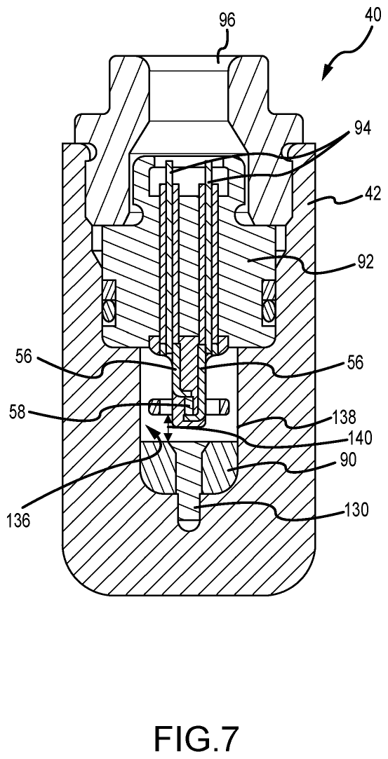

FIG. 7 is a schematic cross-sectional view of an embodiment of the helm resonator sensor 40 in which the flow diverter 90 is arranged at least partially within the flow line 110. In the illustrated embodiment, the inlet and outlet flow line 110 passages are not visible. As shown, the channels 94 extend through the feedthrough 92, thereby providing electrical power to the electrodes 56 coupled to the piezo helm resonator 58. As a result, the piezo helm resonator 58 may resonate within the fluid cavity 116, which enables measurement of a variety of fluid properties.

The illustrated embodiment further includes the gap thickness 136 that substantially surrounds at least a portion of the piezo helm resonator 58. Additionally, the distance 140 is further illustrated in FIG. 7. As shown, at least a portion of the piezo helm resonator 58 is separated vertically from the flow diverter 90. This separation reduces the effects of acoustic reflections from the walls of the flow line on the electrical admittance spectra of the helm resonator.

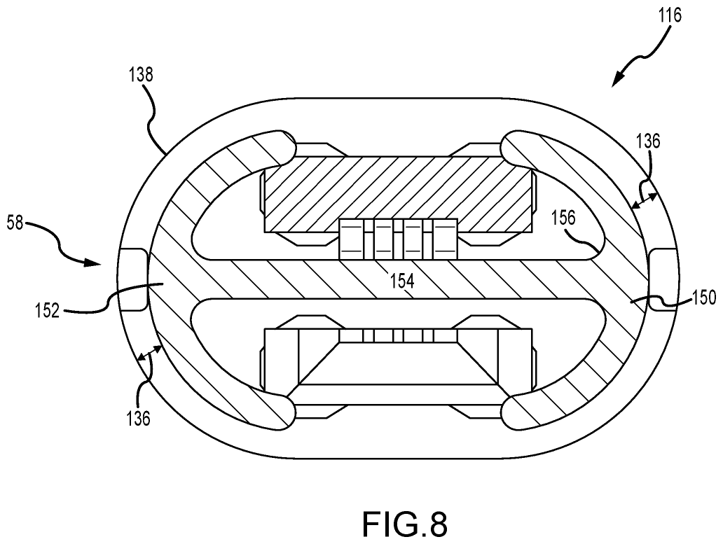

FIG. 8 is a schematic top plan view of an embodiment of the piezo helm resonator 58 arranged within the fluid cavity 116. As described above, the annular wall 138 forms at least part of a barrier of the fluid cavity 116. In the illustrated embodiment, the gap thickness 136 is substantially equal between the curvature of tines 150, 152 of the piezo helm resonator 58 and the annular wall 138. In various embodiments, curvature of the annular wall 138 conforms to the curvature of the tines 150, 152, thereby enabling the uniform gap thickness 136. This gap thickness 136 enables density and viscosity measurements that are essentially linear functions of resonance frequency shifts due to the elimination of any sensitivity of frequency shifts to changes in fluid sound speed. As used herein, sound speed refers to a speed at which acoustic waves propagate through a particular fluid.

The illustrated piezo helm resonator 58 includes a strain bar 154 coupling the tines 150, 152 together. In various embodiments, a fillet 156 or other connection is positioned between the strain bar 154 and the tines 150, 152. As illustrated, the fillet 156 is curved, which reduces stresses between the strain bar 154 and the tines 150, 152. It should be appreciated, and will be described further below, that the strain bar 154 may also be referred to as a Poisson strain bar and, moreover, may not have a uniform thickness across its length. That is, various portions of the straight bar 154 may be adjusted or otherwise formed in order to reduce weight, induce a certain resonance frequency, create and/or eliminate a certain sensitivity to fluid properties, and the like. Accordingly, the embodiment illustrated in FIG. 8 should be considered for example purposes only and not be used to limit the general structure of the strain bar 154, and moreover the piezo helm resonator 58.

In operation, the piezo helm resonator 58 may experience an induced resonance displacement along the strain bar 154, causing the tines 150, 152 to vibrate with oscillatory motion in the gap thickness 136. It should be appreciated that the gap thickness 136 may be particularly selected based at least in part on the geometry of the piezo helm resonator 58. For example, in various embodiments, the gap thickness 136 may be approximately one half a width of the tines 150, 152. Furthermore, the gap thickness 136 may be approximately ¼ the width of the tines 150, 152; approximately ⅓ the width of the tines 150, 152; approximately ⅝ the width of the tines 150, 152; approximately ¾ the width of the tines 150, 152; or any other reasonable size.

In the illustrated embodiment, the piezo helm resonator 58 design is based on the combination of a Poisson strain bar 154 and a symmetric pair of vibratory helm-geometry tines 150, 152. The helm tines 150, 152 are excited by placing an electrical voltage across opposing faces of the transverse thickness of the piezoelectric strain bar 154 segment to develop an oscillatory contraction/expansion of the bar thickness. Due to the Poisson's ratio effect, this through-thickness oscillatory motion develops a longitudinal oscillatory displacement along the length of the bar 154 that excites the helm tines 150, 152 into resonance response. Due to the helm geometry of the tines 150, 152, the frequency and bandwidth of the resonance response is dependent upon the visco-acoustic properties of the fluid surrounding the tines. This characteristic can be used to determine the visco-acoustic properties of the fluid, namely fluid density and viscosity, from measurement of the electrical admittance spectrum (50-60 kHz) on the piezoelectric resonator driving circuit.

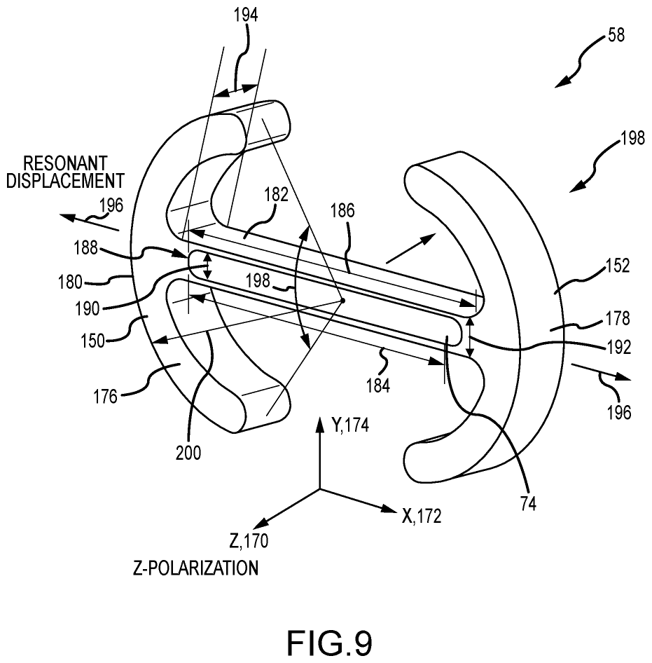

FIG. 9 is an isometric perspective view of an embodiment of the piezo helm resonator 58. For clarity with the below discussion, the piezo helm resonator 58 will be discussed with reference to the illustrated coordinate system, where a Z-axis 170, an X-axis 172, and a Y-axis 174 are used for reference only. In the illustrated embodiment, a first face 176 corresponds to the Z-axis, a second face 178 corresponds to the X-axis on the tine 152, a third face 180 corresponds to the X-axis on the tine 150, and a fourth face 182 corresponds to the Y-axis. It should be appreciated that the first and fourth faces 176, 182 have opposite faces that are not visible in the illustrated embodiment.

In various embodiments, the piezo helm resonator 58 is formed using a monolithic construction to negate coupled modes, thereby creating substantially a classic response of the electrical admittance spectra. Furthermore, production costs may be reduced due to the ease of forming a singular piece, as well as coating the piece. In various embodiments, the piezo helm resonator 58 may include an electro-silver plating over the piezo electric wafer. However, it should be appreciated that the piezo helm resonator 58 may be formed from any reasonable material that may be induced to resonate by the opposing electrical voltages.

Turning to the first face 176, the resonator electrode 74 extends an electrode length 184, which is less than a bar length 186, in the illustrated embodiment. However, it should be appreciated that in various embodiments the electrode length 184 may be substantially equal to the bar length 186. In various embodiments, a surface area 188 of the resonator electrode 74 may determinate, at least in part, a magnitude of an emitted signal. Accordingly, a larger surface area 188 may induce more movement of the piezo helm resonator 58, as well as improve a signal/noise ratio associated with the helm resonator sensor 40. The resonator electrode 74 also includes an electrode height 190, which is less than a bar height 192. However, in various embodiments, the heights 190, 192 may be substantially equal. It should be appreciated that certain terms such as height, thickness, width, and the like may be used interchangeably to describe various properties of the piezo helm resonator 58. These terms may be interchangeable due to the three dimensional coordinate system and the point of view that the piezo helm resonator 58 is viewed. For instance, a height (substantially up and down relative to the page) may be viewed as a width (substantially left to right relative to the page) based on the perspective at which the piezo helm resonator 58 is viewed. Furthermore, it should be appreciated that the resonator electrode 74 is also mounted on the opposing face that is not visible in the present view because the applied electrical voltages induce strain along the Z-axis 170 in the strain bar 154, which drives lateral movement along the X-axis 172.

While the illustrated embodiment includes the straight bar 154 with a substantially uniform height 192 across the length 186, it should be appreciated that the height 192 may vary at different points along the length 186. That is, the height 192 may be larger at certain points along the length 186. This may be done in order to reduce weight, accommodate various design parameters, improve the strength of the piezo helm resonator 58, or to increase the surface area 188.

Turning to the fourth face 182, a thickness 194 of the straight bar 154 is represented. In various embodiments, the thickness 194 and the height 192 are substantially equal. However, they may not be equal. For example, the height 192 may be larger or the thickness 194 may be larger. Furthermore, the illustrated thickness 194 is substantially constant along the length 184 of the strain bar 154. However, it should be appreciated that the thickness 194 may vary along the length of the bar 184.

In operation, the ratio of the length 186 to the thickness 194 (e.g., the X-axis distance/the Z-axis distance) will govern the magnitude of the longitudinal motion along the X-axis 172 represented by the arrows 196. That is, as the length 186 increases, the displacement along the X-axis also increases. Changes to the combination of helm tine arc length and strain bar longitudinal length 186 are used to change the resonance mode shape and subsequently the sensitivity of the resonance frequency and bandwidth to fluid visco-acoustic properties. Accordingly, the dimensions of the piezo helm resonator 58 may be particularly selected, and adjusted relative to one another, to induce different resonance responses. This may be desirable where an array of piezo helm resonators 58 is deployed. One or more of the array may be designed differently to induce different responses over a wide range of fluids. As a result, many different measurements may be acquired and then compared to determine various fluidic properties.

Turning to the tines 150, 152 represented by the third face 180 and the fourth face 182, the illustrated tines 150, 152 are substantially equal and are coupled to the strain bar 154 at substantially mid points. In various embodiments, the tine 150, 152 have an arc 198. The arc 198 in the illustrated embodiment is approximately 160 degrees. The tines 150, 152 further include a radius 200. It should be appreciated that the arc 198 and/or the radius 200 may be particularly selected based on the length 186. For example, the length 186 may be equal to approximately 2 times the radius 200. However, this relationship is only an example and, in other embodiments, different relationships between the various dimensions may be established based on operating conditions or the like. In various embodiments, the helm geometry shape of the tines 150, 152 increases the depth of fluid investigation by developing a resonance pressure gradient over the perimeter of the tines 150, 152 that extends more than 1 mm into the fluid. The longitudinal motion of the strain bar 154 acts along a line (e.g., the X-axis 172) connecting the two dynamical stationary points of the resonance mode shape of the helm tines 150, 152. This allows the strain bar 154 to be used as a mounting interface that has relatively minimal effect on the resonance frequency and mode shape of the resonator.

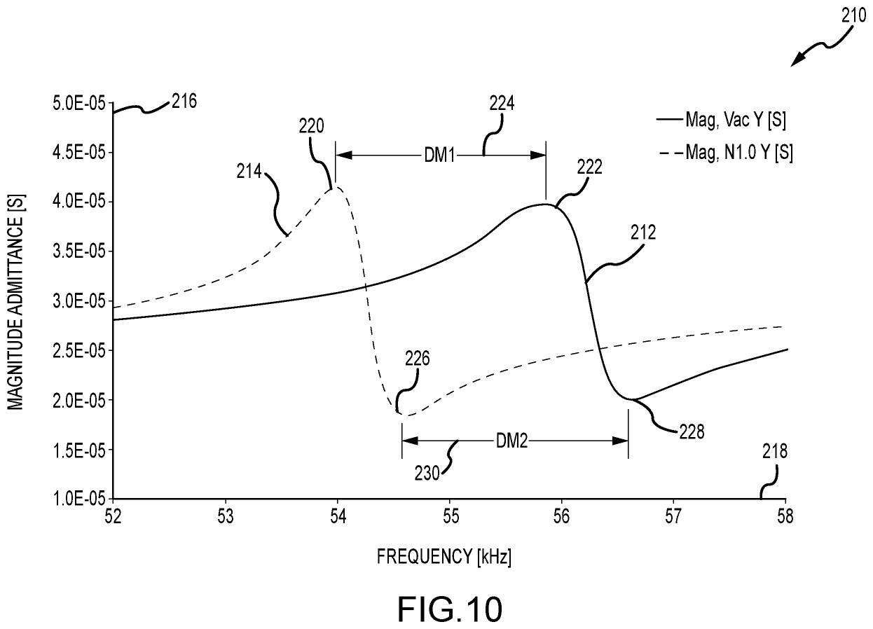

FIG. 10 is a graphical representation 210 of a first admittance 212 and a second admittance 214. Certain features regarding calculating and utilizing admittance spectrum are described in U.S. patent application Ser. No. 14/705,523, filed Nov. 10, 2016, which is hereby incorporated by reference in its entirety. In the illustrated embodiment, the first admittance 212 may be equal to an air admittance, such as a laboratory experiment conducted within atmospheric air. It should be appreciated that the second admittance 214 appears shifted or offset from the first admittance 212. In various embodiments, a basic set of parametric functions of the components of the admittance spectra are assumed to determine intrinsic sensitivity of the helm resonator to changes in fluid properties. The parametric functions assumed may be comprised of the shifts in resonance frequency of components of the admittance spectrum from a reference spectrum where the sensor operates in air.

In the illustrated embodiment, a y-axis 216 of the graphical representation 210 corresponds to a magnitude admittance measured in siemens (S) while an x-axis 218 corresponds to a frequency in kilohertz (kHz). In the illustrated embodiment, a peak 220 of the second admittance 214 is offset from a peak 222 of the first admittance 212 by a distance 224, designated DM1. This shift may be referred to as a designation of the frequency shift of the resonance magnitude peak 220 from the reference air admittance spectrum 212. The illustrated embodiment also includes a trough 226 of the second admittance 214 that is offset from a trough 228 of the first admittance 212 by a distance 230, designated DM2. This shift may be utilized in order to calculate densities for a variety of fluids based on calibration with known laboratory fluid samples.

In various embodiments, best-fit trends of the density prediction with the helm resonator sensor 40 follow a substantially linear relation with the shift of the admittance magnitude resonance peak 220. The relationship may be given by:

ρ=a₁·DM1, (1)

where ‘DM1’ designates the frequency shift of the resonance magnitude peak from the reference air admittance spectrum, as described above and a₁ is a coefficient deduced from laboratory experiments using calibrated fluid samples. Accordingly, density may be calculated by evaluating a shift between the peaks and multiplying this shift by the coefficient. In various embodiments, admittance spectrum for a variety of different fluids may be analyzed and correlated in laboratory conditions, stored within a database, and utilized in operation with the helm resonator sensor 40 to analyze densities in real or near-real time (e.g., without significant delay). As described above, in various embodiments, an array of sensors 40 may be utilized to measure densities along a fluid flow path. It should be appreciated that the second admittance 214 may be obtained from the piezo helm resonator 58 in combination with a variety of instrumentation and sensor systems.

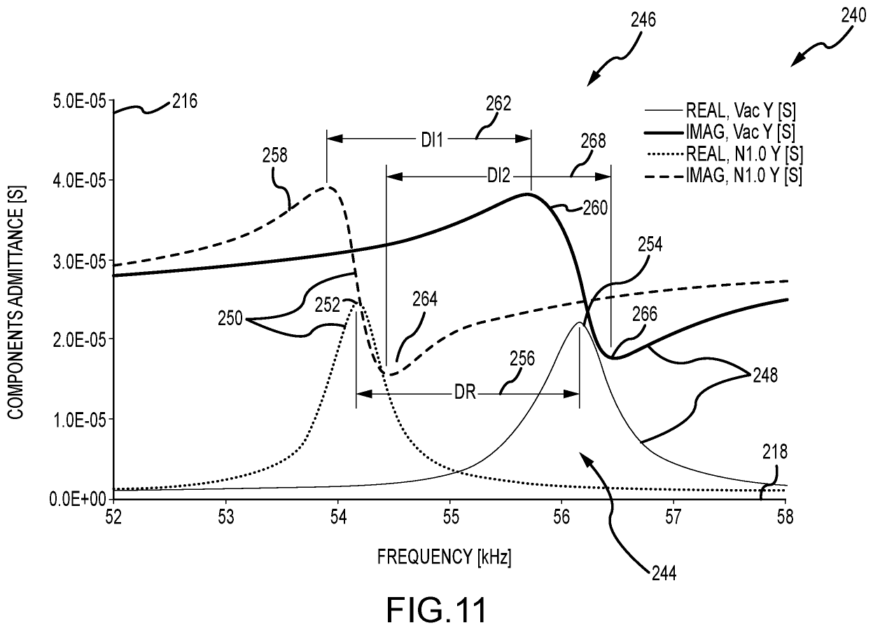

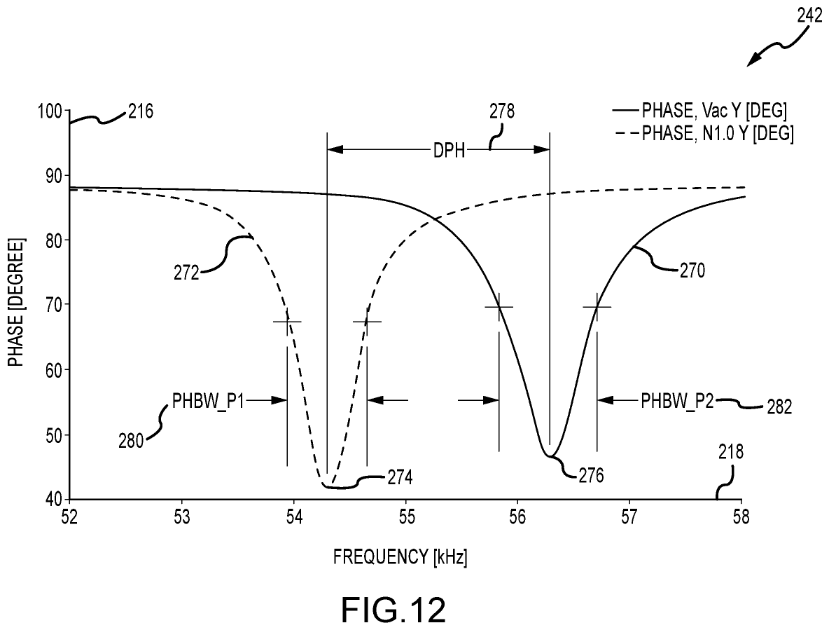

As described in detail above, the helm resonator sensor 40 may be utilized in order to conduct a variety of measurements using a single sensor (or an array of sensors, each conducting multiple measurements). In various embodiments, viscosity may be determined utilizing the helm resonator sensor 40. FIGS. 11 and 12 are graphical representations 240, 242 of components admittance (S) and phase, respectively. When evaluating viscosity, it is important to note that the admittance spectrum will include both a real portion 244 and an imaginary portion 246. These components may be evaluated separately in order to determine viscosity of fluid within the fluid cavity 116.

Turning to FIG. 11, the graphical representation 240 in the y-axis 216 and the x-axis 218 representing admittance (S) and frequency (kHz), respectively, and includes a first admittance 248 and a second admittance 250. Similarly to the admittance spectrum of FIG. 10, the first admittance 248 may correspond to an air admittance while the second admittance 250 may correspond to data obtained from the helm resonator sensor 40 immersed in a fluid of interest. The second admittance 250 includes a first peak 252 in the real portion 244 that is offset from a first peak 254 of the first admittance 248 by a distance 256, designated DR. The second admittance 250 further includes a second peak 258 in the imaginary portion 246 that is offset from a second peak 260 of the first admittance 248 by a distance 262, designated DI1 Additionally, the second admittance 250 also includes a trough 264 in the imaginary portion 246 that is offset from a trough 266 of the first admittance 248 by a distance 268, designated DI2. As will be described below, these offsets may be utilized to develop a relationship between electrical admittance and viscosity to enable calculations using the helm resonator sensor 40.

FIG. 12 illustrates the graphical representation 242 where the y-axis 216 corresponds to phase (degrees) and the x-axis 218 corresponds to frequency (kHz). The illustrated representation 242 includes a first phase spectrum 270 and a second phase spectrum 272. Similar to the above, the first phase spectrum 270 may correspond to an air electrical admittance phase spectrum while the second phase spectrum 272 corresponds to a fluid electrical admittance phase spectrum obtained via the helm resonator sensor 40. In the illustrated embodiment, the second phase spectrum 272 includes a trough 274 that is offset from a trough 276 of the first phase spectrum 270 by a distance 278, designated DPH. Additionally, the illustrated first phase spectrum 270 includes a range corresponding to bandwidth 280, designated PHBW_p1 and the second phase spectrum 272 includes a range correspond to bandwidth 282, designated PHBW_p2. As will be described below, these distances and the bandwidths may be utilized to calculate viscosity of the fluid.

In various embodiments, the best-fit trends of the Helm Resonator sensitivity with changes in fluid viscosity (ν) may be described by two multivariable models, one for ν<20 cPs and the second for 20<ν<270 cPs. The multivariable model for the low viscosity region ν<20 cPs is given by:

Log(ν₁)=b₀+b₁·DI2²+b₂·Log(DM2)²+b₃·Log(DPH)², (2)

where, as described above, ‘DI2’ designates the frequency shift of the imaginary component valley (e.g., trough), ‘DM2’ designates the frequency shift of the magnitude valley (e.g., trough), and ‘DPH’ designates the frequency shift of the admittance resonance phase valley (e.g., trough) from the reference air admittance spectrum. The numerical values for the b₁ coefficients may be particularly deduced from laboratory experiments using calibrated fluid samples.

The multivariable model for the higher viscosity region 20<ν<270 cPs is given by:

Log(ν₂)=c₀+c₁·DI2+c₂·Log(DM1)²+c₃·DPHBW, (3)

where, as described above, ‘DI2’ designates the frequency shift of the imaginary component valley, ‘DM1’ designates the frequency shift of the magnitude peak, and ‘DPHBW’ designates the change in resonance frequency bandwidth between the phase inflection points. The numerical values for the c₁ coefficients may be particularly selected, as described above.

As illustrated, there are two distinct viscosity models, represented by Equations (2) and (3). In order to make the decision as to which of the viscosity models to implement, a less accurate viscosity model may serve as a method for discrimination between the low viscosity and high viscosity regimes about the transition point of ν=20 cPs. In various embodiments, the best-fit trends for a rough transition detection of the Helm Resonator sensitivity with changes in fluid viscosity over the joint range 0.3<ν<220 cPs could be described by one multivariable model given by:

Log(ν₁₋₂)=d₀+d₁·DI2⁴+d₂·DI2DI1+d₃·Log(DI1)², (4)

where ‘DI2’ designates the frequency shift of the imaginary component valley, ‘DI2DI1’ designates the change in the bandwidth between the imaginary component peak and valley, and ‘DI1’ designates the frequency shift of the imaginary component peak. The numerical values for the dᵢ coefficients may be deduced from laboratory experiments using calibrated fluid samples, as described above. In various embodiments, the transition detection model determines whether the cavity fluid has a viscosity above or below 20 cPs, which enables selection of the appropriate model for determining viscosity.

As described above, in various embodiments the piezo helm resonator 58 includes the EM coils 72 on each side. These coils 72 may be arranged on opposing faces of the lateral cross section of the resonator 58 to develop an electromagnetic dipole field in the fluid cavity 116 in order to obtain an electromagnetic impedance spectroscopy for the fluid sample. As would be understood, in various embodiments the dielectric constants of water, rock, and oil may be used to estimate water content in a downhole formation. In various embodiments, this information may be utilized to determine the conductivity of the fluid sample. Furthermore, the conductivity may further be used, at least in part with a machine learning method, in order to provide a quantitative assessment of contamination.

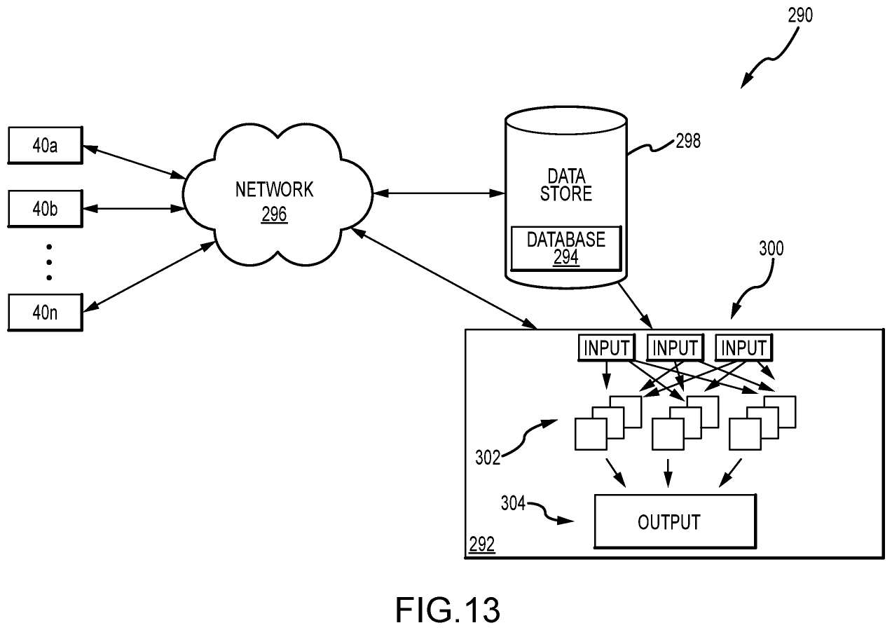

FIG. 13 is a block diagram of an embodiment of a machine learning system 290 that may be utilized with embodiment of the present disclosure. Embodiments of the present disclosure may utilize machine learning techniques to associate specific electromagnetic impedance spectroscopy with specific fluid mixtures, thus enabling not only fluid property identification but fluid mixture characterization. The machine learning techniques may include one or more neural networks (e.g., convolutional neural networks, fully connected neural networks, recurrent neural networks, etc.) to analyze how data related to electromagnetic impedance spectroscopy may relate to ground truth information related to fluid mixture characterization. In other words, the machine learning method may obtain information identifying fluid mixture characterizations based on electromagnetic impedance spectroscopy (e.g., a ground truth) and thereafter “learn” how different electromagnetic impedance spectroscopy information may correlate to that fluid characterization, as well as others. In certain embodiments, the machine learning techniques may incorporate one or more open source machine learning libraries, such as TensorFlow, scikit-learn, Theano, Pylearn2, NuPIC, and the like.

It should be appreciated that in certain embodiments the machine learning system 290 may be incorporated into a control system associated with the wireline/drilling system 10. The control system may include one or more processors and memories. The memories may store instructions that, when executed by the processors, perform one or more functions. Additionally, in embodiments, the machine learning system 290 may be associated with a remote server having a processor (e.g., central processing unit, graphics processing unit, etc.) and a memory. In the illustrated embodiment, the machine learning system 290 includes a machine learning module 292 that may be trained using known information (e.g., a ground truth) such as a database 294. In this training step, the machine learning module 292 is utilized to correlate data between fluid mixtures and their associated electromagnetic impedance spectroscopy. It should be appreciated that the machine learning module 292 may be trained using any variety of methods, such as back propagation, clustering, or any other reasonable methods.

As shown in FIG. 13, data from the helm resonator sensor(s) 40 may be transmitted to a network 296, for example via a network communication system, such as the Internet or the like. The network 296 may include the database 294 and/or be in communication with the database 294, which may be stored in a data store 298. The data store 298 may be utilized for training purposes for the machine learning module 292 or to transmit data to the machine learning module 292 for evaluation. It should be appreciated that data may also be transmitted directly to the machine learning module 292 from the network 296.

The illustrated embodiment of the machine learning module 292 includes a convolutional neural network that takes input 300 through one or more convolutional steps 302, which may include pooling, non-linearization (e.g., ReLu), filtering, and the like. The result of the convolutional steps 302 may be further processed to from an output 304 based on one or more parameters of the machine learning module 292. For instance, if the machine learning module 292 is trained to identify fluid mixture properties, such as a percentage of drilling mud in the fluid, then the machine learning module 292 may output information indicative of different percentages of fluids within the fluid cavity, a predefined characterization (e.g., mud-heavy, mud-light, etc.), or a percentage of mud. In certain embodiments, this may be referred to as identification of the contamination of the fluid.

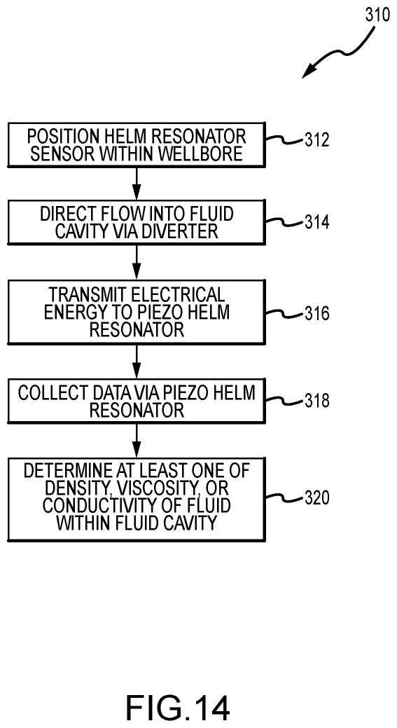

FIG. 14 is a flow chart of an embodiment of a method 310 for collecting and analyzing data utilizing the piezo helm resonator 40. It should be understood that, for any process described herein, that there can be additional, alternative, or fewer steps performed in similar or alternative orders, or concurrently, within the scope of the various embodiments unless otherwise specifically stated. The illustrated method 310 includes positioning the piezo helm resonator 40 within the wellbore 18 (block 312). In various embodiments, the piezo helm resonator 40 may be associated with (e.g., installed on) the wireline/drilling string 14. However, it should be appreciated that the piezo helm resonator 40 may be installed on a production string that is not associated with drilling operations. The method 310 further includes directing flow into the fluid cavity 116 via the flow diverter 90 (block 314). As described above, in various embodiments the flow diverter 90 reduces acoustic reflections within the fluid cavity 116, thereby enabling accurate measurements irrespective of sound speed for the specific fluid. Furthermore, the flow diverter 90 enables circulation throughout the fluid cavity 116, which enables measurement of different portions of the fluid flow over a period of time. Accordingly, information related to how fluid properties change may be obtained.

Thereafter, electrical energy is transmitted to the piezo helm resonator 40 (block 316). For example, the cables 50 may energize at least one of the coil 72 and the resonator electrode 74. As will be appreciated from the above discussion, energizing the piezo helm resonator 40 induces strain along the strain bar 154 to drive longitudinal displacement along the x-axis 172. Such displacement may be used to determine the electrical admittance associated with the fluid within the fluid cavity 116. Data may be collected as the piezo helm resonator 40 is excited (block 318). As described above, in various embodiments this data may be stored onboard the wireline/drill string 14, transmitted uphole, and/or transmitted offsite for evaluation. Next, at least one of the density, viscosity, or conductivity of the fluid within the fluid cavity 116 is determined (block 320), for example using the equations presented above. In this manner, fluidic properties may be obtained from the fluid within the fluid cavity 116. Moreover, in various embodiments, the density, viscosity, and conductivity of the fluid may be obtained using information obtained from a single sensor.

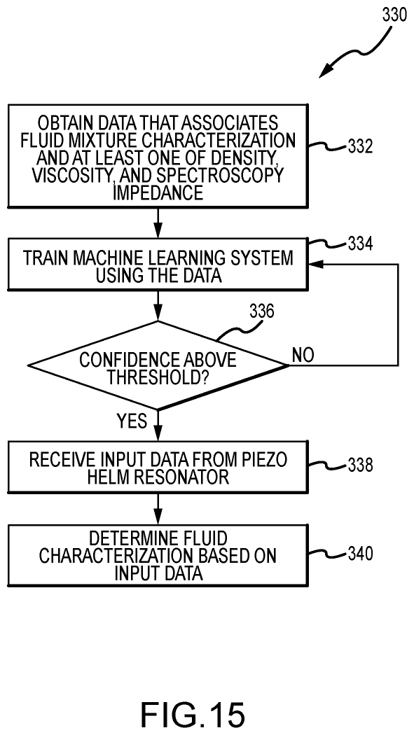

FIG. 15 is a flow chart of an embodiment of a method 330 for training and utilizing a machine learning system for determining a fluid characterization. In the illustrated embodiment, training data is obtained (block 332). The training data may include information that associates a fluid mixture characterization (e.g., contamination) with at least one of density, viscosity, or spectroscopy impedance of the fluid. This information may be obtained from laboratory testing, field data, and the like. Next, a machine learning system is trained using the training data (block 334). Various machine learning systems and training methods were discussed above. Upon completion of the training, a confidence level of the system may be determined. The confidence level may be associated with a likelihood that the machine learning system provides a correct response when presented with an inquiry. The confidence level may be checked against a threshold (block 336). If the confidence level is below the threshold, then additional data may be obtained for further training. If the confidence level is above the threshold, the machine learning system may be presented with input data from the piezo helm resonator (block 338). In various embodiments, the data may be related to density, viscosity, and/or spectroscopy impedance. It should be appreciated that the data from the piezo helm resonator may be raw data, filtered data, or data that has been manipulated and adjusted for input into the machine learning system. Thereafter, a fluid characterization is determined (block 340). In various embodiments, the fluid characterization may be referred to as contamination, for example, and may provide an indication as to the amount of non-productive fluid is in the sample. Non-productive fluid may refer to drilling fluid, fracturing fluid, acids, washes, and the like. Accordingly, the machine learning system may be utilized to analyze one or more fluidic properties to characterize the fluid.

The foregoing disclosure and description of the disclosed embodiments is illustrative and explanatory of the embodiments of the invention. Various changes in the details of the illustrated embodiments can be made within the scope of the appended claims without departing from the true spirit of the disclosure. The embodiments of the present disclosure should only be limited by the following claims and their legal equivalents.

1. A system for measuring a fluidic property of a fluid, the system comprising:
a housing including an opening, the opening extending longitudinally along an axis of the housing;
a flow passage extending through the passage, the flow passage intersecting the opening;
a flow diverter arranged at an intersection between the opening and the flow passage, the flow diverter directing a fluid flowing through the flow passage into a fluid cavity formed at least partially in the opening; and
a piezo helm resonator arranged within the fluid cavity, the piezo helm resonator electrically coupled to a power supply that transmits electrical energy to at least one resonator electrode arranged on the piezo helm resonator, wherein the piezo helm resonator resonates within the fluid cavity when electrically energized by the power supply.

2. The system of claim 1, further comprising:
a retainer coupled to the housing and extending at least partially into the opening; and
a feedthrough arranged between the retainer and the piezo helm resonator, wherein the retainer secures the feedthrough within the housing.

3. The system of claim 2, wherein the fluid cavity it at least partially defined by an annular wall of the housing, the feedthrough, and the flow diverter.

4. The system of claim 1, wherein the piezo helm resonator further comprises:
a strain bar; and
a pair of tines coupled to opposite ends of the strain bar, the tines having an arc such that strain across a transverse face of the strain bar generates a resonance response from the pair of tines.

5. The system of claim 4, wherein the piezo helm resonator further comprises:
at least one electromagnetic spectroscopy coil arranged along the strain bar, wherein the electromagnetic spectroscopy coil receives electrical energy from the power supply.

6. The system of claim 1, further comprising:
a gap thickness that surround the piezo helm resonator within the fluid cavity, the gap thickness providing a void space between at least a portion of an annular wall of the fluid cavity and piezo helm resonator.

7. The system of claim 1, further comprising:
a machine learning system communicatively coupled to the helm resonator sensor, the machine learning system receiving data from the helm resonator sensor to determine a fluid classification for a fluid positioned within the fluid cavity.

8. The system of claim 1, wherein the flow diverter further comprises:
a leading edge arranged to face an upstream portion of the flow passage; and
a trailing edge arranged to face a downstream portion of the flow passage;
wherein the leading edge drives the fluid flow into the fluid cavity to circulate around the piezo helm resonator, the leading edge being arranged at an angle relative to the flow passage to induce stagnant fluid in the fluid cavity to exit the fluid cavity along the training edge.

9. A method for determining a fluid property, the method comprising:
positioning a helm resonator sensor within a wellbore;
directing a flow of fluid into a fluid cavity of the helm resonator sensor;
transmitting electrical energy to a piezo helm resonator within the fluid cavity;
collecting data associated with at least one fluid property via the piezo helm resonator; and
determining the at least one fluid property based at least in part on the collected data.

10. The method of claim 9, further comprising:
measuring an electrical admittance of the piezo helm resonator within the fluid;
comparing a resonance frequency offset between the measured admittance and a reference admittance; and
determining the at least one fluid property based at least in part on the resonance frequency offset.

11. The method of claim 9, wherein the flow of fluid is directed into the fluid cavity with a flow diverter, the flow diverter inducing circulation of fluid within the fluid cavity.

12. The method of claim 9, further comprising:
measuring an electrical admittance of the piezo helm resonator within the fluid;
determining a real portion of the admittance;
determining an imaginary portion of the admittance;
determining a first frequency offset between the measured real portion and a reference real portion;
determining a second frequency offset between the measured imaginary portion and a reference imaginary portion; and
determining the at least one fluid property based at least in part on the first and second resonance frequency offsets.

13. The method of claim 9, further comprising:
providing ground truth data corresponding to the at least one fluid property, the ground truth data correlating the collected data and the at least one fluid property;
training a neural network using the ground truth data; and
inputting the collected data into the trained neural network.

14. The method of claim 9, wherein the at least one fluid property comprises a density, a viscosity, a conductivity, a fluid classification, or a combination thereof.

15. The method of claim 9, wherein the helm resonator sensor is an array of helm resonator sensors.

16. A method for determining at least one fluid property, the method comprising:
obtaining electrical admittance data from a downhole tool, the electrical admittance data being associated with a fluid in a wellbore;
obtaining reference electrical admittance data for a reference fluid;
comparing the electrical admittance data to the reference electrical admittance data;
determining a set of admittance resonance frequency offsets from the reference electrical admittance data; and
determining the at least one fluid property based at least in part on the determined set of admittance resonance frequency offsets.

17. The method of claim 16, further comprising:
providing ground truth data corresponding to the at least one fluid property, the ground truth data correlating the electrical admittance data and the at least one fluid property;
training a neural network using the ground truth data; and
inputting the collected data into the trained neural network.

18. The method of claim 16, further comprising:
determining a real portion of the electrical admittance data;
determining an imaginary portion of the electrical admittance data;
determining a real portion of the reference electrical admittance data;
determining an imaginary portion of the reference electrical admittance data;
determining a resonance frequency real offset between the real portion of the electrical admittance data and the real portion of the reference electrical admittance data;
determining a resonance frequency imaginary offset between the imaginary portion of the electrical admittance data and the imaginary portion of the reference electrical admittance data; and
determining the at least one fluid property based at least in part on the real and imaginary offsets.

19. The method of claim 16, further comprising:
energizing a piezo helm resonator positioned in contact with the fluid.

20. The method of claim 16, further comprising:
measuring conductivity using electromagnetic spectroscopy coils via the downhole tool.
